(19) United States
(12) Patent Application Publication
(10) Pub. No.: US 2023/0190986 A1
(43) Pub. Date: Jun. 22, 2023

(54) DISCHARGE DEVICE

(71) Applicant: SHARP KABUSHIKI KAISHA, Sakai City (JP)

(72) Inventors: SATOSHI OKANO, Sakai city (JP); NOBUYUKI OHE, Sakai City (JP)

(21) Appl. No.: 17/971,629

(22) Filed: Oct. 23, 2022

(30) Foreign Application Priority Data
Nov. 17, 2021 (JP) 2021-187088

(51) Int. Cl. A61L 9/22; H05H 1/24

(52) U.S. Cl. CPC A61L 9/22; H05H 1/24; H05H 2245/15

(57) ABSTRACT

A discharge device releases a discharge product into the air. The discharge device includes a housing, a discharge electrode, and two protectors. The discharge electrode protrudes from the housing. The discharge electrode releases a discharge product into the air by discharging. The protectors are members separate from the housing. The two protectors are provided on either side of the discharge electrode. Each of the protectors includes a pair of supports and an electrode protector. The pair of supports is provided on the housing. The support extends in a direction separating from the housing. An end edge of each support is located at a position further separated from the housing than a position of a tip portion of the discharge electrode. The electrode protector connects an end edge side of one of the pair of supports to an end edge side of the other of the pair of supports.

### CROSS-REFERENCE TO RELATED APPLICATIONS

This application claims the benefit of priority to Japanese Patent Application No. 2021-187088 filed on Nov. 17, 2021. The entire contents of the above-identified application are hereby incorporated by reference.

### BACKGROUND

### Technical Field

The disclosure relates to a discharge device.

WO 2020/013143 discloses a discharge device configured to release a discharge product by discharge from an electrode.

### SUMMARY

The discharge device of WO 2020/013143 can release a high concentration of discharge product into the air. However, there is room for improving the structure to release an even higher concentration of discharge product.

The disclosure has been made in view of the problem described above, and an object thereof is to provide a discharge device that can release a high concentration of discharge product.

According to an aspect of the disclosure, a discharge device releases a discharge product into the air. The discharge device includes a housing, a discharge electrode, and two protectors. The discharge electrode protrudes from the housing. The discharge electrode releases a discharge product into the air by discharging. The protectors are members separate from the housing. The two protectors are provided on either side of the discharge electrode. Each of the protectors includes a pair of supports and an electrode protector. The pair of supports is provided on the housing. Each of the pair of supports extends in a direction separating from the housing. An end edge of each support is located at a position further separated from the housing than a position of a tip portion of the discharge electrode. The electrode protector connects an end edge of one the pair of supports to an end edge of the other of the pair of supports. The tip portion of the discharge electrode is visually recognizable when the discharge electrode is viewed from a front direction and a side direction of the housing.

According to the disclosure, a discharge device that can release a high concentration of discharge product can be provided.

### BRIEF DESCRIPTION OF DRAWINGS

The disclosure will be described with reference to the accompanying drawings, wherein like numbers reference like elements.

FIG. 1 is a perspective view illustrating an electronic apparatus including a discharge device according to an embodiment of the disclosure.

FIG. 2 is a cross-sectional view of an electronic apparatus including a discharge device according to the embodiment of the disclosure.

FIG. 3 is a cross-sectional view of a holder to which a discharge device according to the embodiment of the disclosure is attached.

FIG. 4 is a perspective view of a discharge device according to the embodiment of the disclosure.

FIG. 5 is a cross-sectional view illustrating a discharge device according to the embodiment of the disclosure.

### DESCRIPTION OF EMBODIMENTS

Embodiments of the disclosure will be described hereinafter with reference to the accompanying drawings. Note that, in the drawings, the same or equivalent components are denoted by the same reference signs and description thereof will not be repeated.

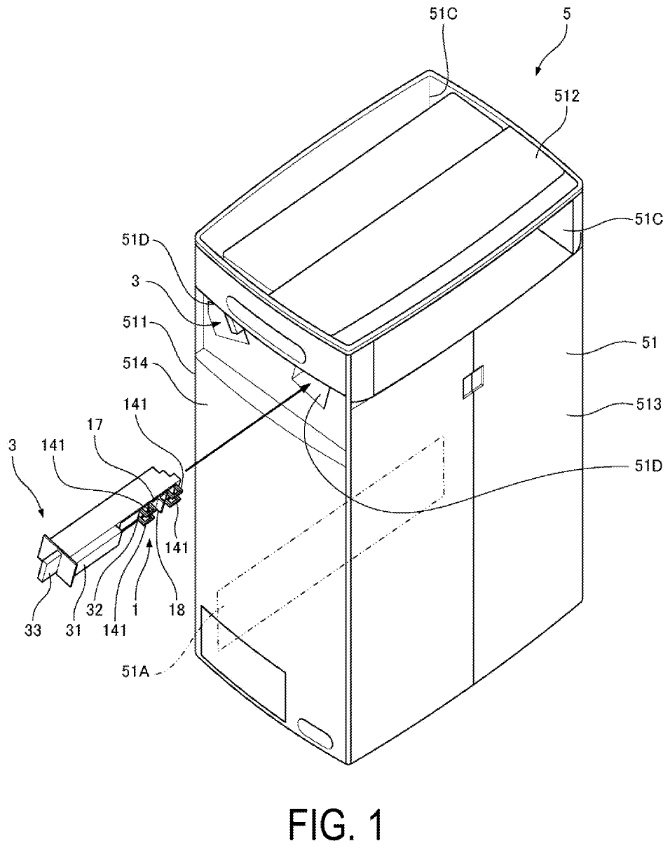

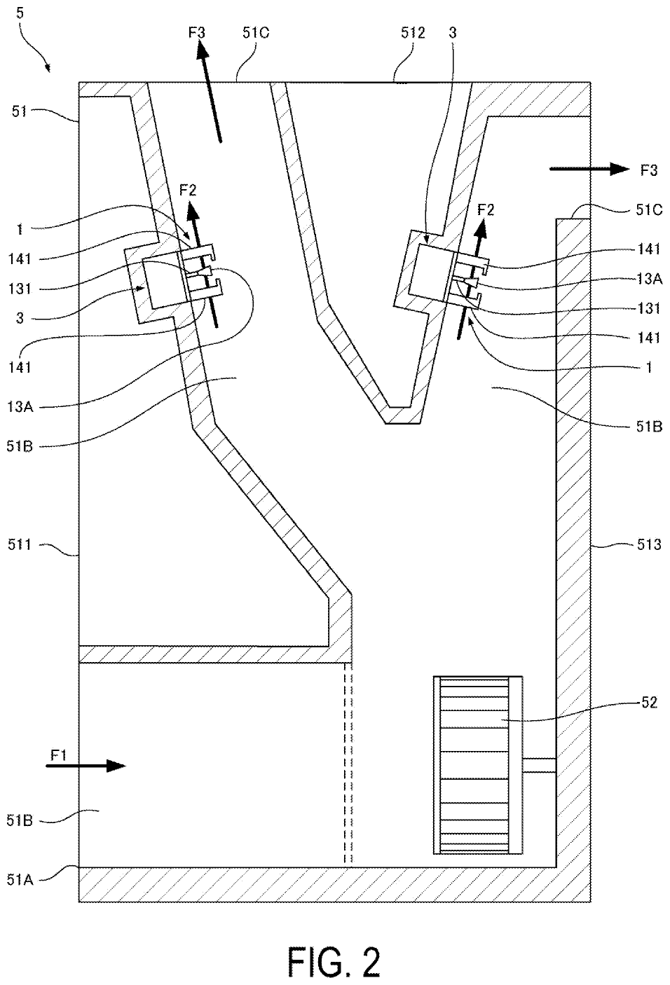

Before describing a discharge device 1 of the disclosure, an electronic apparatus 5 including the discharge device 1 of the disclosure will be described with reference to FIGS. 1 and 2. FIG. 1 is a perspective view illustrating the electronic apparatus 5 including the discharge device 1 according to an embodiment of the disclosure. FIG. 2 is a cross-sectional view of the electronic apparatus 5 including the discharge device 1 according to the embodiment of the disclosure.

The electronic apparatus 5 illustrated in FIGS. 1 and 2 includes a blower function and a function of releasing a discharge product. In addition to the functions described above, the electronic apparatus 5 may also include a function of changing the physical property of air.

The blower function is a function of intaking air around the electronic apparatus 5 into the electronic apparatus 5, and sending out the in-taken air to the outside of the electronic apparatus 5. The function of releasing a discharge product includes, for example, a function of suppressing the action of bacteria by generating ions or free radicals. The function of changing the physical property of air includes, for example, a function of removing particles in the air. Specifically, the electronic apparatus 5 having the blower function and the function of releasing a discharge product into the air is a discharge product generator such as an ion generator or an ozone generator. As a discharge product generator with the function of removing particles contained in the air, the electronic apparatus 5 is an air purifier with a discharge product generating function. The electronic apparatus 5 may include, in addition to an air purifier with a discharge product generating function, a humidifying air purifier with a discharge product generating function, and a humidifying/dehumidifying air purifier with a discharge product generating function.

The electronic apparatus 5 includes amain body 51, a holder 3, and the discharge device 1. The discharge device 1 is attached to the holder 3, and the holder 3 is disposed inside the main body 51, which has a box shape.

Components (not illustrated) for operating the electronic apparatus 5 are provided in the main body 51. The components include a power supply cable (not illustrated) provided outside the main body 51, and a feeder connector (not illustrated) provided inside the main body 51. A voltage is applied through the feeder connector.

As illustrated in FIG. 2, an inlet port 51A that allows the outside and the inside of the main body 51 to communicate is formed in a main body rear surface 511. The inlet port 51A communicates with a ventilation passage 51B provided inside the main body 51. The ventilation passage 51B is continuous with an outlet port 51C provided in a main body top surface 512 and a main body front surface 513. The outlet port 51C communicates with the ventilation passage 51B and the outside of the main body 51.

A fan 52 is installed inside the ventilation passage 51B. The fan 52 is, for example, a sirocco fan. The fan 52 rotates to generate an airflow F1 of intaking air present outside the main body 51 into the ventilation passage 51B through the inlet port 51A, an airflow F2 of the sucked air passing through the ventilation passage 51B toward the outlet port 51C, and an airflow F3 of sending out the air from the outlet port 51C to the outside of the main body 51.

As illustrated in FIG. 1, a holder insertion port 51D that allows the inside and the outside of the main body 51 to communicate is formed in a main body side surface 514. The space inside the main body 51 communicating with the holder insertion port 51D communicates with the ventilation passage 51B illustrated in FIG. 2. A feeder connector is disposed in this space.

A voltage is applied to the discharge device 1 through the feeder connector, so that a discharge product is released. Examples of the discharge product released by the discharge device 1 include ions and free radicals. Examples of the ions include a positive ion (for example, H⁺(H₂O)ₘ, where m is any integer), a negative ion (for example, O₂⁻(H₂O)ₙ, where n is any integer), or a combination of these ions. Examples of the free radicals include a hydroxyl radical (−OH), a hydrogen radical (—H), an oxygen radical (—O), a hydroperoxyl radical (—HO₂), hydrogen peroxide (H₂O₂), and ozone (O₃). The discharge product has, for example, an effect of suppressing the action of bacteria and suppressing harmful effects of the bacteria on the human body.

The discharge device 1 is attached to the holder 3. The discharge device 1 attached to the holder 3 is inserted into the main body 51 through the holder insertion port 51D. As illustrated in FIG. 2, the discharge device 1 inserted into the main body 51 is connected to the feeder connector, and is arranged such that part of the discharge device 1 is exposed to the ventilation passage 51B. The holder 3, in which the discharge device 1 is arranged as described above, is held in the main body 51 . Avoltage is applied to the discharge device 1 connected to the feeder connector. The discharge device 1 releases a discharge product into the airflow F2 passing through the ventilation passage 51B. As a result, the discharge product included in the airflow F2 passing through the ventilation passage 51B is sent out through the outlet port 51C to the outside of the main body 51, and suppresses the action of bacteria present outside the main body 51.

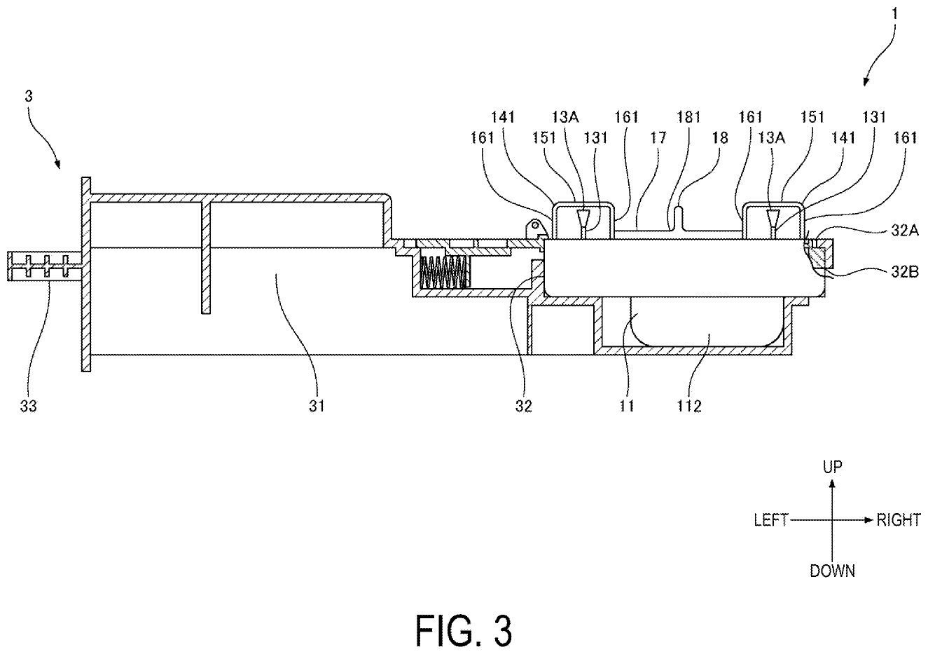

Referring to FIGS. 1 to 3, the holder 3 to which the discharge device 1 of the disclosure is to be attached will be described below. FIG. 3 is a cross-sectional view of the holder 3 to which the discharge device 1 according to the embodiment of the disclosure is attached.

As illustrated in FIG. 3, the holder 3 includes a holder base 31, a housing portion 32, and a holder handle 33. Hereinafter, in the description of the holder 3, the directions illustrated in FIG. 3 are defined as up-down and left-right directions for convenience. That is, the longitudinal direction of the holder 3 is defined as the left-right direction. The direction orthogonal to the left-right direction is defined as the up-down direction.

The holder base 31 is a portion for setting the position of the housing portion 32, where the discharge device 1 is housed and attached, with respect to the main body 51 illustrated in FIG. 2. The housing portion 32 is provided at a right end portion of the holder base 31. By appropriately setting the length of the holder base 31 in the left-right direction, the discharge device 1 housed in and attached to the housing portion 32 is partially disposed in the ventilation passage 51B illustrated in FIG. 2, and the discharge device 1 is connected with the feeder connector.

As described above, the discharge device 1 is attached to the housing portion 32. The housing portion 32 has a box shape. A discharge device housing port 32A open in the up-down direction is formed in the upper portion of the housing portion 32. The discharge device housing port 32A allows the inside and the outside of the housing portion 32 to communicate. The discharge device 1 passes through the discharge device housing port 32A to move into or out of the housing portion 32. A connector insertion port 32B open in the left-right direction is formed in a right end portion of the housing portion 32. The connector insertion port 32B allows the inside and the outside of the housing portion 32 to communicate. The discharge device 1 housed in the housing portion 32 is connected with the feeder connector via the connector insertion port 32B.

The holder handle 33 is connected to a left end portion of the holder base 31. The holder handle 33 is a portion that allows a user, who is an operator of the electronic apparatus 5 illustrated in FIGS. 1 and 2, to hold the holder 3. When holding the holder handle 33, the user can insert the housing portion 32 and the holder base 31 of the holder 3 into the holder insertion port 51D illustrated in FIG. 1 and perform an operation of arranging the housing portion 32 inside the main body 51.

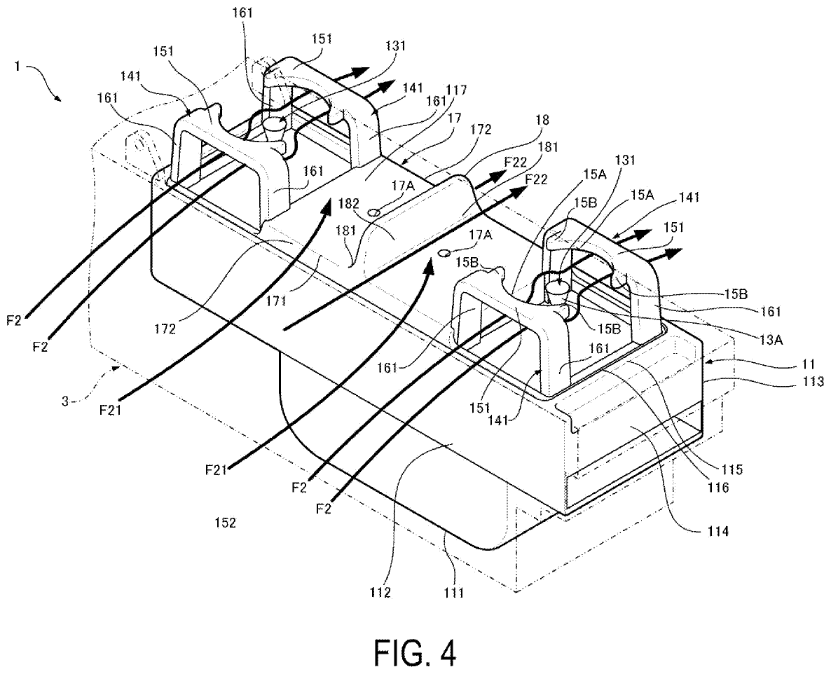

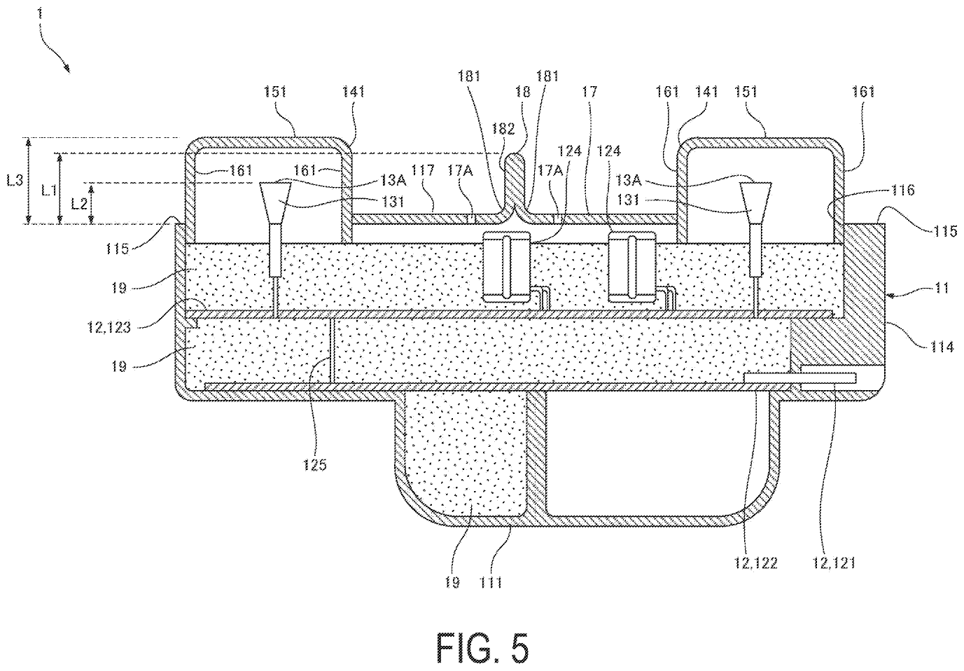

The discharge device 1 according to the embodiment of the disclosure will be described with reference to FIG. 1 to FIG. 5. FIG. 4 is a perspective view illustrating the discharge device 1 according to the embodiment of the disclosure. FIG. 5 is a cross-sectional view of the discharge device 1 according to the embodiment of the disclosure.

As illustrated in FIGS. 4 and 5, the discharge device 1 includes a housing 11, electrical components 12, two discharge electrodes 131 and 131, a protector 141 provided for each of the discharge electrodes 131, a coupling portion 17, a partition wall 18, and a filling resin 19.

As illustrated in FIG. 4, the housing 11 has a bottom surface 111, a front surface 112 connected to the bottom surface 111, a rear surface 113 connected to the bottom surface 111 and opposing the front surface 112, two side surfaces 114, and a top surface 115 opposing the bottom surface 111. A portion of the housing 11 surrounded by the bottom surface 111, part of the front surface 112, part of the rear surface 113, and parts of the two side surfaces 114 is housed in the housing portion 32 of the holder 3. When the holder 3 housing the housing 11 is attached to the main body 51, the front surface 112 of the housing 11 faces the upstream side of the airflow F2 passing through the ventilation passage 51B illustrated in FIG. 2. When the holder 3 housing the housing 11 is attached to the main body 51, a portion extending from the top surface 115 including the protector 141 slightly toward the bottom surface 111 is exposed to the ventilation passage 51B.

As illustrated in FIG. 5, the top surface 115 of the housing 11 includes an opening 116 that allows the inside and the outside of the housing 11 to communicate. The electrical components 12 for operating the discharge electrodes 131 and 131 are provided inside the housing 11. The discharge device 1 of the present embodiment includes a power-receiving connector 121, a control substrate 122, and a high voltage substrate 123 as the electrical components 12.

The power-receiving connector 121 is a pin disposed in a substantially perpendicular direction with respect to the side surface 114 of the housing 11. The power-receiving connector 121 is connected to the feeder connector so that a voltage formed in the main body 51 illustrated in FIGS. 1 and 2 can be supplied to the control substrate 122 connected to the power-receiving connector 121.

The control substrate 122 is disposed inside the housing 11. The control substrate 122 has a wide plate shape. The control substrate 122 supplies a high voltage to the high voltage substrate 123 connected via a wiring line 125.

The high voltage substrate 123 is disposed at a position closer to the top surface 115 relative to the control substrate 122 inside the housing 11. The high voltage substrate 123 has a wide plate shape, and includes a plurality of electrical elements 124 on a surface facing the top surface 115. The high voltage substrate 123 generates a high voltage by a component (not illustrated) mounted on the control substrate 122, and supplies the high voltage to each of the two discharge electrodes 131 and 131 connected to the high voltage substrate 123.

The discharge electrode 131 has a tip portion 13A protruding outward of the housing 11. Thus, as illustrated in FIG. 2, when the holder 3 housing the discharge device 1 is attached to the main body 51, the tip portion 13A of the discharge electrode 131 is located in the ventilation passage 51B. Due to discharge by the tip portion 13A located in the ventilation passage 51B, the discharge electrode 131 releases a discharge product into the air present in the ventilation passage 51B. When the airflow F2 passes through the ventilation passage 51B, the discharge electrode 131 releases the discharge product into the airflow F2. The discharge electrode 131 is formed of a conductive material such as a metal, carbon fiber, conductive fiber, or conductive resin. The discharge electrode 131 of the present embodiment includes a plurality of linear conductors bundled together to form a brush shape. Note that the shape of the discharge electrode 131 is not limited to a brush shape, and may be any shape such as a rod shape, a needle shape, a linear shape, a fibrous shape, or a plane shape.

As illustrated in FIG. 5, the discharge device 1 includes the two discharge electrodes 131 and 131. When both of the discharge products released from the discharge electrodes 131 and 131 are positive or negative ions, the discharge product (for example, positive ions) released from one discharge electrode 131 has a polarity opposite to that of the discharge product (for example, negative ions) released from the other discharge electrode 131. When discharge products having mutually opposite polarities come into contact with each other, these discharge products (in this case, positive and negative ions) are neutralized. Accordingly, by reducing the amount of discharge products to be neutralized, the discharge device 1 may release a high concentration of discharge product. The discharge products are neutralized in the air because discharge products having mutually opposite polarities (positive and negative ions) are attracted to each other. The discharge product released into the air may be absorbed by a substance having a low insulation property, and thereafter may disappear inside the substance.

As illustrated in FIG. 4, the protector 141 is a member separate from the housing 11. The protector 141 has a simple structure compared to a structure in which the protector 141 and the housing 11 are integrated. As a result, the protector 141 can be more easily manufactured than when the protector 141 and the housing 11 are integrated. One protector 141 is provided on either side of one discharge electrode 131. The two protectors 141 and 141 are provided such that one is arranged on the front surface 112 side relative to the discharge electrode 131 and the other is arranged on the rear surface 113 side relative to the discharge electrode 131 on the housing 11. In other words, one of the protectors 141 is provided on the upstream side and one of the protectors 141 is provided on the downstream side, relative to the discharge electrode 131, along the ventilation passage 51B. The protectors 141 and 141 are disposed so as not to block the airflow F2 traveling toward the discharge electrode 131. In the above description, the protectors 141 and 141 are provided on the front surface 112 side and the rear surface 113 side relative to the discharge electrode 131, but the protectors 141 and 141 may be provided at any positions in the housing 11 provided that they are arranged on either side of the discharge electrode 131 and do not block the airflow F2 traveling toward the discharge electrode 131. For example, the protectors 141 and 141 may be provided on the housing 11 on either side of the discharge electrode 131 in a direction from the side surface 114 side.

When the protectors 141 and 141 described above are provided, the airflow F2 passing through the ventilation passage 51B keeps traveling toward the discharge electrode 131 without being obstructed by the protectors 141 and 141. As a result, the discharge electrode 131 can release the discharge product into the airflow F2 passing through the ventilation passage 51B. The discharge product released from the discharge electrode 131 into the airflow F2 passing through the ventilation passage 51B is dispersed from the discharge device 1 before coming into contact with the discharge product having a different polarity released from the other discharge electrode 131.

The protector 141 includes an electrode protector 151 having a rod shape and a pair of supports 161 and 161 each having a rod shape, thereby forming a gate shape. The pair of supports 161 and 161 protrudes from the inside to the outside of the housing 11 through the opening 116. The pair of supports 161 and 161 extends in a direction separating from the housing 11 with a portion attached to the inside of the housing 11 as a start edge. An end edge of the pair of supports 161 and 161 is located at a position further separated from the housing 11 than a position of the tip portion 13A of the discharge electrode 131. The electrode protector 151 connects an end edge side of one support 161 to an end edge side of the other support 161. With this, the electrode protector 151 is provided extending in a direction parallel to the top surface 115 and being distanced from the housing 11, so as to protect the tip portion 13A of the discharge electrode 131. Since the two protectors 141 and 141 having the structure described above are attached to the housing 11, the tip portion 13A of the discharge electrode 131 is visually recognizable from the front direction and the side direction of the housing 11. When the protector 141 is provided, the electrode protector 151 prevents contact with the discharge electrode 131 due to incorrect operation or the like when the discharge device 1 is attached to the holder 3. In the above description, the protector 141 is formed in a gate shape in which the pair of supports 161, 161 is connected via the electrode protector 151, but the protector 141 may have any structure in which the pair of supports 161, 161 and the electrode protector 151 are integrated or discrete components connected to each other. In addition, the protector 141 is formed in a gate shape connecting the rod-shaped electrode protector 151 and the pair of rod-shaped supports 161 and 161, but the protector 141 may be formed in an arch shape without combining the rod-shaped components provided that the tip portion 13A of the discharge electrode 131 is visually recognizable when viewed from the front direction and the side direction of the housing 11.

The electrode protector 151 includes a curved portion 15A and a protrusion 15B.

The curved portion 15A is formed at a portion of the electrode protector 151 on the discharge electrode 131 side when the electrode protector 151 is viewed in the direction of the top surface 115 of the housing 11. The curved portion 15A is formed in a curved shape in which the center portion along the length direction of the electrode protector 151 is further distanced from the electrode protector 151 along the direction of the airflow F2 in the ventilation passage 51B than both end portions of the electrode protector 151 along the length direction. In other words, the curved portion 15A surrounds the discharge electrode 131 in a curved shape along the circumferential direction of the discharge electrode 131 while maintaining a space at an equivalent distance from the discharge electrode 131. When the curved portion 15A is provided, in comparison to an electrode protector formed in a shape not including the curved portion 15A (such as a linear shape), the distance from the tip portion 13A of the discharge electrode 131 to the electrode protector 151 is large especially at the center portion of the electrode protector 151 along the length direction. As a result, the amount of discharge product that arrives at the electrode protector 151 and is absorbed by the electrode protector 151 is reduced as compared to the electrode protector formed in a shape not including the curved portion 15A (such as a linear shape), so that the amount of discharge product released into the airflow F2 passing through the ventilation passage 51B increases. That is, the discharge device 1 can release a high concentration of discharge product.

The protrusion 15B protrudes from an end portion of the curved portion 15A in the direction of the airflow F2 passing through the ventilation passage 51B. In other words, the protrusion 15B protrudes in a direction approaching the discharge electrode 131. When the electrode protector 151 is provided, the curved portion 15A of the electrode protector 151 maintains a constant distance from the discharge electrode 131. In the electrode protector 151, the distance between the protrusion 15B and the discharge electrode 131 is small in comparison to a case where the electrode protector 151 does not include the protrusion 15B. As a result, the protrusion 15B prevents a situation where a user's finger on the discharge device 1 moves beyond the protector 141 and comes into contact with the discharge electrode 131 when the discharge device 1 is attached to the holder 3.

The electrode protector 151 of the protector 141 is supported by the two supports 161 and 161. With this configuration, the protector 141 does not block the airflow F2 flowing toward the discharge electrode 131. In other words, the protector 141 surrounds the discharge electrode 131 while ensuring a space through which the airflow F2 passes through the ventilation passage 51B. As a result, the discharge electrode 131 can reliably release the discharge product into the airflow F2 passing through the ventilation passage 51B, as described above.

The coupling portion 17 connects one support 161 in each of the two protectors 141 protecting one discharge electrode 131 and one support 161 in each of the two protectors 141 protecting the other discharge electrode 131 to each other. When the coupling portion 17 is provided, the four protectors 141, 141, 141, 141 are attached to the housing 11 together. As a result, less work is needed to attach the four protectors 141, 141, 141, 141 to the housing 11 compared to a case where the coupling portion 17 is not provided.

The coupling portion 17 has a plate shape, and includes the top surface 117 and a pair of end surfaces 172, 172 continuous with the top surface 117. One of the pair of end surfaces 172 and 172 is located on the upstream side of the airflow F2 passing through the ventilation passage 51B, and the other of the pair of end surfaces 172 and 172 is located on the downstream side of the airflow F2 passing through the ventilation passage 51B. The coupling portion 17 is disposed such that the top surface 117 is arranged along the airflow F2 passing through the ventilation passage 51B illustrated in FIG. 2. The coupling portion 17 is attached to the housing 11 so that part of the coupling portion 17 in the thickness direction thereof including the top surface 117 projects to the outside of the housing 11. That is, the coupling portion 17 is attached to the housing 11 such that the airflow F2 passing through the ventilation passage 51B comes into contact with the coupling portion 17.

The coupling portion 17 includes a first curved surface 171. The first curved surface 171 connects the end surface 172 and the top surface 117. The airflow F2 passing through the ventilation passage 51B is in contact with the first curved surface 171 on the upstream side of the airflow F2. The airflow F2 in contact with the first curved surface 171 is guided by the first curved surface 171.

The first curved surface 171 guides a first airflow F21 guided by the first curved surface 171 so that the airflow separates from the top surface 117 of the coupling portion 17 in the thickness direction of the coupling portion 17. With this, the discharge product traveling from one of the discharge electrodes 131 and 131 toward the other is included in the first airflow F21 guided by the first curved surface 171, and is separated from the coupling portion 17 and dispersed. As a result, the amount of discharge product traveling from one of the discharge electrodes 131 and 131 toward the other is reduced and the amount of discharge product to be neutralized is reduced, as compared to a case where the coupling portion 17 does not include the first curved surface 171.

The coupling portion 17 also includes the first curved surface 171 on the end surface 172 on the downstream side of the airflow F2. With this, the housing 11 can be attached even when the attachment positions of the end surfaces 172 on the upstream and downstream sides with respect to the housing 11 are reversed. Therefore, the attachment direction of the coupling portion 17 to the housing 11 need not be identified.

The coupling portion 17 closes the opening 116 of the housing 11 to cover the electrical components 12 disposed inside the housing 11. The coupling portion 17 serves as a wall that physically and visually separates the inside and the outside of the housing 11. As a result, the coupling portion 17 prevents foreign matter from entering the housing 11. Further, in the case of the discharge device 1 including the coupling portion 17, the electrical components 12 are not visually recognizable from the outside of the housing 11, and thus the discharge device 1 has a better design compared to a configuration where the electrical components 12 are visually recognizable from the outside.

The coupling portion 17 is coupled to the supports 161 at a base end of each of the supports 161. With the above-mentioned coupling portion 17, the length from the coupling portion 17 to the electrode protector 151 is longer compared to a configuration where the supports 161 are not coupled at the base ends of the supports 161. This makes it possible to manufacture, with small dimensions, a protector 141 having a structure that causes the airflow F2 passing through the ventilation passage 51B to avoid a path through which the airflow F2 passes while making contact with the discharge electrode 131, in comparison with a configuration where the coupling portion 17 is not coupled to the base ends of the supports 161.

The partition wall 18 protrudes from the coupling portion 17 toward the ventilation passage 51B, and is formed along the direction of the airflow F2 passing through the ventilation passage 51B. The partition wall 18 separates the two discharge electrodes 131 and 131 from each other. When the partition wall 18 is provided, it is possible to inhibit the movement of discharge products of opposite polarities that are attracted by each other, as described above. As a result, the amount of discharge product neutralized in the air is reduced as compared to a configuration where the partition wall 18 is not provided.

As illustrated in FIG. 5, a length L1 from the top surface 115 of the housing 11 to the top of the partition wall 18 in a direction protruding from the housing 11 is greater than or equal to a length L2 from the housing 11 to the point of the tip portion 13A of the discharge electrode 131 in a direction in which the partition wall 18 protrudes from the housing 11. In addition, the length L1 from the top surface 115 of the housing 11 to the top of the partition wall 18 is less than or equal to a length L3 from the top surface 115 of the housing 11 to the tip surface of the electrode protector 151 of the protector 141 in the direction in which the partition wall 18 protrudes from the housing 11.

When the partition wall 18 is provided, the shortest path from one of the two discharge electrodes 131 and 131 to the other has a shape that bypasses the partition wall 18. Further, from the relationship between the length L1, the length L2, and the length L3 described above, as long as the electronic apparatus 5 has a size that allows the protector 141 to be disposed in the ventilation passage 51B, the discharge device 1 may be attached to the electronic apparatus 5 without any trouble regardless of whether the partition wall 18, the discharge electrodes 131, and the like are in a protruding state. Because the shortest path has the bypass shape described above, the amount of discharge product to be neutralized decreases compared to a configuration where the shortest path from one of the two discharge electrodes 131 and 131 to the other is linear. Whether the discharge device 1 can be attached to the electronic apparatus 5 depends on the size of the protector 141. Accordingly, the discharge device 1 can be attached to the electronic apparatus 5 including the ventilation passage 51B having the minimum size necessary to protect the discharge electrode 131.

As illustrated in FIG. 4, a side surface 182 of the partition wall 18 and the top surface 117 of the coupling portion 17 are connected via a second curved surface 181. When the partition wall 18 is provided, part of the airflow F2 along the partition wall 18 is guided by the second curved surface 181 at a boundary portion between the partition wall 18 and the top surface 117. As a result, the turbulence of a second airflow F22 guided by the second curved surface 181 is small as compared to a configuration where the side surface 182 of the partition wall 18 and the top surface 117 of the coupling portion 17 are connected by a corner portion and no second curved surface 181 is provided. Thus, the discharge product is unlikely to stagnate near the partition wall 18. This makes it possible to prevent discharge products of mutually different polarities from coming into contact with each other due to the above-mentioned stagnation.

As illustrated in FIG. 5, when the inside of the housing 11 is filled with the filling resin 19 and then the filling resin 19 is cured, the electrical components 12, the discharge electrodes 131, the protectors 141, the coupling portion 17, and the partition wall 18 are bonded to the housing 11.

The coupling portion 17 preferably has a through-hole 17A extending through the coupling portion 17 in the thickness direction. The through-hole 17A serves as an air vent hole when the housing 11 is filled with the filling resin 19 and the components described above are bonded. With this, when the coupling portion 17 is bonded to the housing 11, air present in the space between the filling resin 19 and the coupling portion 17 passes through and leaves from the through-hole 17A. As a result, when performing the filling and bonding, the coupling portion 17 can be appropriately bonded to the housing 11 without an excessive increase in the pressure in the space between the filling resin 19 and the coupling portion 17. Examples of the filling resin 19 include an epoxy resin.

The coupling portion 17 preferably has two through-holes 17A. One through-hole 17A is formed on either side of the partition wall 18 of the coupling portion 17. With this configuration, when the coupling portion 17 is bonded to the housing 11, air is more likely to leave the space between the filling resin 19 and the coupling portion 17 compared to a configuration where one through-hole 17A is provided. As a result, the coupling portion 17 can be more easily bonded to the housing 11 compared to a configuration where one through-hole 17A is provided.

The two through-holes 17A are each preferably formed at a position closer to the partition wall 18 than to the discharge electrode 131. With this configuration, the two through-holes 17A are less noticeable due to the presence of the partition wall 18. As a result, the electrical components 12 disposed inside the housing 11 are unlikely to be visually recognizable through the through-holes 17A as compared to a configuration where the through-holes 17A are formed at positions separated from the partition wall 18. Accordingly, the discharge device 1, in which the two through-holes 17A are formed at positions closer to the partition wall 18 than to the discharge electrodes 131 and 131, has a good design.

More preferably, the two through-holes 17A are each formed at such a position that the electrical components 12 disposed inside the housing 11 are not visually recognizable through the through-holes 17A. With this configuration, the electrical components 12 disposed inside the housing 11 are not visually recognizable through the through-holes 17A. Accordingly, the discharge device 1 has a good design when compared to a configuration where the electrical components 12 are visually recognizable from the outside.

Preferably, the protectors 141, the coupling portion 17, and the partition wall 18 have an integral structure. With the above structure, the protectors 141, the coupling portion 17, and the partition wall 18 are attached to the housing 11 at the same time. As a result, the protectors 141, the coupling portion 17, and the partition wall 18 can be easily attached to the housing 11 compared to a non-integrated structure.

The embodiments of the disclosure have been described above with reference to the accompanying drawings. However, the disclosure is not limited to the embodiments described above, and the disclosure can be implemented in various modes without departing from the gist thereof. For ease of understanding, the drawings schematically illustrate each component as a main constituent, and the thickness, length, number, spacing, and the like of each component illustrated are different from the actual thickness, length, number, and spacing for convenience of drawing preparation. Further, the material, shape, dimensions, and the like of each component described in the embodiments described above are examples and are not particularly limited, and various modifications can be made within a range that does not substantially deviate from the configuration of the disclosure.

### INDUSTRIAL APPLICABILITY

The disclosure provides a discharge device, and the provided discharge device has industrial applicability.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

1. A discharge device, comprising:
a housing;
a discharge electrode protruding from the housing and configured to release a discharge product into air by discharging; and
two protectors being members separate from the housing and provided on either side of the discharge electrode,
wherein each of the protectors includes
a pair of supports provided on the housing, each of the pair of supports extending in a direction separating from the housing and having an end edge located at a position further separated from the housing than a position of a tip portion of the discharge electrode, and
an electrode protector connecting an end edge side of one of the pair of supports to an end edge side of the other of the pair of supports, and
the tip portion of the discharge electrode is visually recognizable when the discharge electrode is viewed from a front direction and a side direction of the housing.

2. The discharge device according to claim 1,
wherein the electrode protector includes
a curved portion surrounding the discharge electrode in a curved shape while maintaining a space with respect to the discharge electrode, and
a protrusion protruding from an end portion of the curved portion in a direction approaching the discharge electrode.

3. The discharge device according to claim 1,
wherein the electrode protector is supported by the two supports.

4. The discharge device according to claim 1,
wherein the discharge electrode comprises two discharge electrodes,
the discharge electrodes are each protected by the protector, and
the discharge device further includes a coupling portion connecting the support of the protector for protecting one of the discharge electrodes and the support of the protector for protecting the other one of the discharge electrodes.

5. The discharge device according to claim 4,
wherein the coupling portion couples the supports at base ends of the supports.

6. The discharge device according to claim 4,
wherein the coupling portion is formed in a plate shape, and is attached to the housing such that the coupling portion partially projects to an outside of the housing, and
a top surface of the coupling portion disposed to project to the outside of the housing is connected to one end surface disposed at a front side of the housing being distanced in a flow direction of an airflow and the other end surface disposed at a rear side of the housing, via a first curved surface.

7. The discharge device according to claim 4,
wherein the housing includes an opening,
an electrical component configured to operate the discharge electrode is disposed inside the housing communicating with the opening, with the electrical component passing through the opening, and
the opening is closed by the coupling portion to cover the electrical component.

8. The discharge device according to claim 4,
wherein the coupling portion includes a through-hole extending through the coupling portion, and
the coupling portion is bonded to the housing with a resin, with the through-hole functioning as an air vent hole.

9. The discharge device according to claim 4, further comprising:
a partition wall serving as a wall protruding from the coupling portion in a direction separating from the housing and configured to separate one of the two discharge electrodes and the other of the two discharge electrodes from each other.

10. The discharge device according to claim 9,
wherein a length of the partition wall from the housing to a top of the partition wall in a direction in which the partition wall protrudes from the coupling portion is greater than or equal to a length of the discharge electrode from the housing to a tip portion of the discharge electrode in a direction in which the partition wall protrudes from the coupling portion, and is less than or equal to a length of the protector from the housing to the electrode protector in the direction in which the partition wall protrudes from the coupling portion.

11. The discharge device according to claim 9,
wherein a side surface of the partition wall and a top surface of the coupling portion are connected via a second curved surface.

12. The discharge device according to claim 9,
wherein the coupling portion includes a through-hole extending through the coupling portion,
the coupling portion is bonded to the housing with a resin, with the through-hole functioning as an air vent hole,
the coupling portion includes two of the through-holes, and
the two through-holes are disposed on either side of the partition wall.

13. The discharge device according to claim 12,
wherein the through-hole is disposed at a position closer to the partition wall than to the discharge electrode.

14. The discharge device according to claim 9,
wherein the protector, the coupling portion, and the partition wall have an integral structure.
